(19) United States
(12) Patent Application Publication
(10) Pub. No.: US 2021/0065914 A1
(43) Pub. Date: Mar. 4, 2021

(54) DYNAMIC, REAL-TIME, GENOMICS DECISION SUPPORT, RESEARCH, AND SIMULATION

(71) Applicant: SIVOTEC BioInformatics LLC, Boca Raton, FL (US)

(72) Inventors: Pedro L. Martinez, Boca Raton, FL (US); Luis B. Pintado, Boca Raton, FL (US); Klaas Jan J. Wierenga, Jacksonville, FL (US); Nicholas F. Tsinoremas, Miami, FL (US); Christopher C. Mader, Miami Beach, FL (US); Zhijie Jiang, Pembroke Pines, FL (US)

(73) Assignee: SIVOTEC BioInformatics LLC, Boca Raton, FL (US)

(21) Appl. No.: 17/012,824

(22) Filed: Sep. 4, 2020

Related U.S. Application Data
(60) Provisional application No. 62/895,849, filed on Sep. 4, 2019.

(51) Int. Cl. G16H 50/70; G06F 16/11; G16H 50/50; G16H 10/60; G16H 50/20; G16H 70/60; G16H 50/80; G06N 20/00; G06F 30/27; G06F 40/103; G16B 50/00

(52) U.S. Cl. CPC G16H 50/70; G06F 16/116; G16H 50/50; G16H 10/60; G16H 50/20; G16B 50/00; G16H 50/80; G06N 20/00; G06F 30/27; G06F 40/103; G16H 70/60

(57) ABSTRACT

A dynamic, real-time, genomics decision support and simulation system is disclosed. The system receives individual search criteria associated with an individual, and generates and formats a digital file including the individual search criteria into a format suitable for communication, storage, synthesis, analysis, or a combination thereof, by components of the system. The system compares the individual search criteria from the formatted digital file to information from a reference database. Based on the comparing, the system may identify a potential relationship between the individual search criteria and a disease or condition identified in the information from the reference database. The system may present the potential match, along with an analysis relating to the relationship, on a visualization interface on a device associated with the individual.

### CROSS-REFERENCE TO RELATED APPLICATION(S)

This application claims the benefit of U.S. Provisional Patent Application No. 62/895,849 filed Sep. 4, 2019, and is incorporated herein by reference in its entirety.

### FIELD OF THE INVENTION

The present application relates to genomics technologies, simulation technologies, machine learning technologies, artificial intelligence technologies, data aggregation and analysis technologies, database technologies, predictive modeling technologies, big data technologies, and computing technologies, and more particularly, to a system and method for providing dynamic, real-time, genomics decision support, research, and simulation.

### BACKGROUND

In today's technologically-driven society, various systems and methods exist for synthesizing and analyzing various types of data. Notably, however, in various areas of interest, such as genomics science, examining and analyzing the genomic signature of an individual and clarifying the predisposition to disease and health conditions are complex, time consuming, and expensive endeavors. Even though various systems and methods exist for synthesizing data, analyzing data, and examining genomic signatures, such systems and methods are often difficult to utilize and do not provide enough relevant information for decision-makers and users to support making meaningful decisions relating to managing health concerns, preparing regimens for preventing diseases or conditions, and predicting health-related outcomes. Additionally, current technologies and processes often provide irrelevant information, only use limited types of data, require the accessing of data scattered across multiple and disparate data sources, and may be difficult to implement and maintain. Moreover, while current technologies have been utilized to detect existing health conditions or predict possible health outcomes, currently-existing technologies have not provided efficient and optimal means for doing so. As a result, current technologies and processes may be modified and improved so as to provide enhanced functionality and features for users and systems to effectively examine genomic signatures, detect health conditions, conduct predictive modeling, and determine preventative actions for dealing with potential health concerns. Such enhancements and improvements may provide for improved user satisfaction, increased reliability, increased accuracy, increased efficiencies, increased access to meaningful data, substantially-improved decision-making abilities, and increased ease-of-use for users.

### SUMMARY

A system and methods for providing dynamic, real-time, genomics decision support and simulation are disclosed. In particular, the system and accompanying methods provide for an application and technological environment, which utilizes algorithms and various data inputs to determine health conditions, preventive actions for the individual, generate predictive models, conduct simulations and/or perform any other actions of interest. In particular, the system and methods include functionality for receiving individual search criteria associated with an individual from a variety of sources. The system and methods may include processing and converting the received individual search criteria and associated information into a format suitable for communication, storage, synthesis and analysis. Once the individual search criteria and accompanying information is converted and formatted, the system and methods include comparing the individual search criteria and information to other data obtained from one or more reference databases including health and/or other data. Notably, the system and methods may include utilizing any number of mathematical algorithms, machine learning algorithms, and/or artificial intelligence algorithms to perform the comparison. Based on the comparison, the system and methods may include identifying potential relationships (e.g. scientific relationships, potential matches, and/or correlations) between the individual search criteria and a disease, condition, and/or other information from the one or more reference databases.

The system and methods may include conducting various analyses relating to the individual search criteria, the potential relationships (e.g. scientific relationships, potential matches, and/or correlations), and/or other information. Once the analyses are conducted, the system and methods may include providing the individual associated with the individual search criteria (or other designated individual), other users, and/or an automated system with the findings and/or analyses determined by the system and methods. The analyses, the individual search criteria, the potential relationships, and/or any other information may be displayed via an advanced electronic visualization interface (e.g. web-based interface, any type of communication interface, or a combination thereof). In certain embodiments, the analyses, the individual search criteria, the potential relationships, metadata associated with the search criteria, the matches, and/or data from the reference databases may be aggregated with historical individual search criteria and other information stored in a proprietary data warehouse. The proprietary data warehouse may store historical search criteria in a format suitable for analysis of the data by the internal and/or external components of the system. As new information and search criteria are entered into the system, the system and methods may include formatting the analyses, search criteria, in a format for future-reuse by the system, such as for additional system data analysis by-products. Additionally, as new information and search criteria are entered and/or generated by the system, the system and methods may include automatically updating and aggregating such information with historical information previously aggregated in the proprietary data warehouse. Over time, the system and methods increase the amount of information in the reference databases and proprietary data warehouse so that artificial intelligence systems and machine learning systems can provide more effective potential relationship/match determinations over time.

To that end, in one embodiment according to the present disclosure, a system for providing dynamic, real-time, genomics decision support and simulation is disclosed. The system may include a memory that stores instructions and a processor that executes the instructions to perform operations conducted by the system. The system may perform an operation that includes receiving, such as via an interface, individual search criteria associated with an individual. The individual search criteria may include health information, disease information, demographic information, any type of information associated with the user, measured health metrics, keywords, any type of information, or a combination thereof. In certain embodiments, the system may perform an operation that includes generating a digital file including the individual search criteria associated with the individual. The system may proceed to perform an operation that includes formatting the digital file including the individual search criteria into a formatted digital file suitable for communication, storage, synthesis, analysis, or a combination thereof, by components of the system. Once the digital file is formatted, the system may perform an operation that includes comparing the individual search criteria from the formatted digital file to information from a reference database. Based on the comparing, the system may identify a potential relationship between the individual search criteria and a disease, condition, or a combination thereof, identified in the information from the reference database. Furthermore, the system may perform an operation that includes presenting the potential relationship on a visualization interface on a device associated with the individual.

In another embodiment, a method for providing dynamic, real-time, genomics decision support and simulation is disclosed. The method may include utilizing a memory that stores instructions, and a processor that executes the instructions to perform the various functions of the method. In particular, the method may include receiving, such as via an interface, individual search criteria associated with an individual. Additionally, the method may include creating a digital file including the individual search criteria associated with the individual. Also, the method may include converting the digital file including the individual search criteria into a formatted digital file suitable for communication, storage, synthesis, analysis, or a combination thereof, by components of a system implementing the method. The method may then include comparing the individual search criteria from the formatted digital file to information from a reference database. Furthermore, the method may include identifying, based on the comparing, a potential relationship between the individual search criteria and a disease, condition, or a combination thereof, identified in the information from the reference database. Moreover, the method may include displaying the potential relationship on a visualization interface on a device associated with the individual.

According to yet another embodiment, a computer-readable device having instructions for providing dynamic, real-time, genomics decision support and simulation is provided. The computer instructions, which when loaded and executed by a processor, may cause the processor to perform operations including: receiving, via an interface, individual search criteria associated with an individual; generating a digital file including the individual search criteria associated with the individual; converting the digital file including the individual search criteria into a formatted digital file suitable for communication, storage, synthesis, analysis, or a combination thereof, by components of a system implementing the method; comparing the individual search criteria from the formatted digital file to information from a reference database; identifying, based on the comparing, a potential relationship between the individual search criteria and a disease, condition, or a combination thereof, identified in the information from the reference database; and presenting the potential relationship on a visualization interface on a device associated with the individual.

These and other features of the systems and methods for providing dynamic, real-time, genomics decision support and simulation are described in the following detailed description, drawings, and appended claims.

### BRIEF DESCRIPTION OF THE DRAWINGS

FIG. 1 is a schematic diagram of a system for providing dynamic, real-time, genomics decision support and simulation according to an embodiment of the present disclosure.

FIG. 2 is a diagram illustrating various features and components of the system of FIG. 1 including, but not limited to, an artificial intelligence and machine learning system, an electronic visualization tool, a proprietary data warehouse, a plurality of reference databases, and various outputs of the system of FIG. 1.

FIG. 3 is a diagram illustrating a sample screen displayed via a graphical user interface of the system of FIG. 1, which enables a system user to create a unique and private account that will allow the user to access the system according to an embodiment of the present disclosure.

FIG. 4 is a diagram illustrating a sample screen displayed via a graphical user interface of the system of FIG. 1, which enables a user to enter individual search criteria into the system according to an embodiment of the present disclosure.

FIG. 5 is a diagram illustrating an output of an electronic visualization tool of the system of FIG. 1, which provides results derived from artificial intelligence and machine learning system and methods applied to individual search criteria entered into the system.

FIG. 6 is a diagram illustrating a sample screen displayed via a graphical user interface of the system of FIG. 1, which depicts potential diseases and/or conditions determined by the system based on knowledge mined from reference databases in relation to individual search criteria entered into the system of FIG. 1.

FIG. 7 is a diagram illustrating a sample screen displayed via a graphical user interface of the system of FIG. 1, which enables real-time dynamic simulation and research through enabling and/or disabling search filters to individual search criteria, thereby resulting in a newly generated visualization for a desired scenario.

FIG. 8 is a diagram illustrating a sample screen displayed via a graphical user interface of the system of FIG. 1, which enables real-time dynamic simulation and research through enabling and/or disabling search filters to individual search criteria, thereby resulting in a different generated visualization for another desired scenario.

FIG. 9 is a diagram illustrating a sample screen displayed via a graphical user interface which illustrates how the system of FIG. 1 may be used for genetic decision support by accessing deeper layers of disease or condition ontology via interaction with an electronic visualization tool of the system.

FIG. 10 is a flow diagram illustrating a sample method for providing dynamic, real-time, genomics decision support and simulation according to an embodiment of the present disclosure.

FIG. 11 is a schematic diagram of a machine in the form of a computer system within which a set of instructions, when executed, may cause the machine to perform any one or more of the methodologies or operations of the systems and methods for providing dynamic, real-time, genomics decision support and simulation.

### DETAILED DESCRIPTION OF THE INVENTION

A system 100 and methods for providing dynamic, real-time, genomics decision support and simulation are disclosed. In particular, the system 100 and accompanying methods provide for an application and technological environment, which utilizes algorithms and various data inputs to determine health conditions, preventive actions for the individual, generate predictive models, conduct simulations and/or perform any other actions of interest. In particular, the system and methods include functionality for receiving individual search criteria associated with an individual from a variety of sources. The individual search criteria may include, but is not limited to, genomic signature information for an individual, phenotype information for the individual, genetic anomaly information for the individual, DNA information for the individual, keywords associated with the individual and/or a condition associated with the individual, health terms, demographic information, psychographic information, any type of information, any type of media content (e.g. images, audio, video, 3D content, etc.), or a combination thereof. The system 100 and methods may include processing and converting the received individual search criteria and associated information into a format suitable for communication, storage, synthesis and analysis. Once the individual search criteria and accompanying information is converted and formatted, the system 100 and methods include comparing the individual search criteria and information to other data obtained from one or more reference databases 155 including health and/or other data. Notably, the system 100 and methods may include utilizing any number of mathematical algorithms, machine learning algorithms, and/or artificial intelligence algorithms to perform the comparison. Based on the comparison, the system 100 and methods may include identifying potential relationships (e.g. scientific and/or other relationships, matches, and/or correlations) between the individual search criteria and a disease, condition, and/or other information from the one or more reference databases 155.

The system 100 and methods may include conducting various analyses relating to the individual search criteria, the potential relationships, the potential matches, and/or other information. Once the analyses are conducted, the system 100 and methods may include providing the individual associated with the individual search criteria (or other designated individual), other users, and/or an automated system with the findings and/or analyses determined by the system 100 and methods. The analyses, the individual search criteria, the potential relationships, the potential matches, and/or any other information may be displayed via an advanced electronic visualization interface. In certain embodiments, the analyses, the individual search criteria, the potential relationships, the potential matches, metadata associated with the search criteria, the relationships, the matches, and/or data from the reference databases 155 may be aggregated with historical individual search criteria and other information stored in a proprietary data warehouse 204. The proprietary data warehouse 204 may store historical search criteria in a format suitable for analysis of the data by the internal and/or external components of the system. In certain embodiments, the system 100 and methods may also include determining preventative actions for the individual to perform to deal with a condition, execute simulations relating to progression of the condition and/or treatment of the condition, conduct real-time monitoring of the individual, generate predictive models to predict how the individual will progress over time, aggregate research functionality and content, conduct simulations of outbreaks, detect genetic anomalies, generate correlations between and among diseases, detect population shifts in health, and perform and myriad of additional functionality. As new information and search criteria are entered into the system 100, the system 100 and methods may include formatting the analyses, search criteria, in a format for future-reuse by the system 100, such as for additional system data analysis by-products. Additionally, as new information and search criteria are entered and/or generated by the system 100, the system 100 and methods may include automatically updating and aggregated such information with historical information previously aggregated in the proprietary data warehouse 204. Over time, the system and methods increase the amount of information in the reference databases 155 and proprietary data warehouse 204 so that artificial intelligence systems and machine learning systems can provide more effective potential relationship/match determinations over time.

As indicated above, examining the genomic signature of an individual and clarifying the predisposition of the individual to disease and health conditions is complex, time consuming, and expensive. By leveraging artificial intelligence and machine learning systems and methods, the system 100 and methods described herein provide a software and hardware platform defined to conduct dynamic, real-time genomic decision support and simulation functions. The system 100 and methods allow for an accelerated analysis and interactive modeling of genomic sequences from microarrays, exome, custom genomic sequences, full genomes, and/or other-related methods. Artificial intelligence and machine-learning methods provided by the system 100 and methods provide informed and prioritized high-speed filtering and analysis. Machine learning provided by the system 100 and methods optimally navigates the utility of base of data and enhances its search and analytic algorithms with each transaction processed by the system 100 and methods. Moreover, the interactive dynamic visualization generated and outputted using the system 100 and methods creates a unique functional capability for multi-dimensional discovery matching phenotype (observable health conditions) with genotype (objective genetic signature), and other relevant data (environmental, lifestyle, cognitive). The real-time drill-down by the system 100 and methods into existing conditions and related genes and variants provides unlimited possibilities for discovery. The system 100 and methods also provide the basis for simulation of conditions or genes to analyze and predict potential outcomes. The implications for identification of subjects for clinical trials, geneticists, drug discovery, population health management, and related providers and payers are broad and will significantly reduce time, increase quality of discovery, and reduce costs, while continuing to perfect its analysis through increased population dynamics, size, and big data.

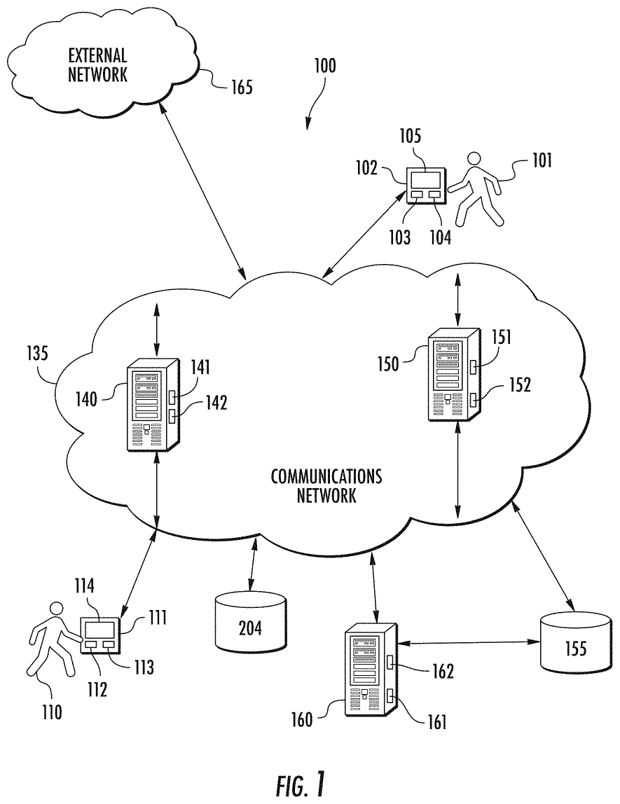

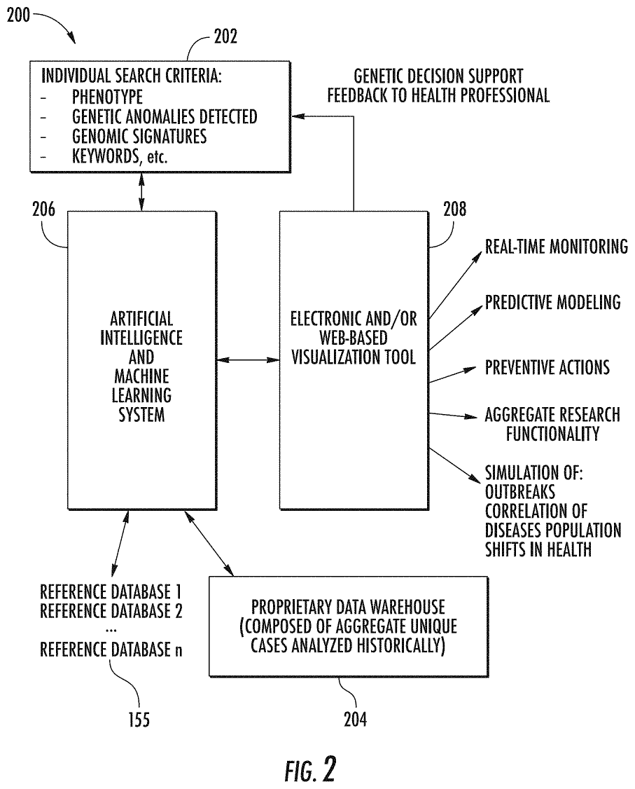

As shown in FIGS. 1-4, a system 100 and method for providing dynamic, real-time, genomics decision support and simulation using artificial intelligence, machine learning, and/or other techniques are disclosed. The system 100 may be configured to support, but is not limited to supporting, data and content services, data aggregation applications and services, genomic analysis technologies, simulation technologies, phenotype and genotype analysis technologies, predictive modeling technologies, big data technologies, health disease and condition analysis technologies, data synthesis applications and services, data analysis applications and services, computing applications and services, cloud computing services, internet services, satellite services, telephone services, software as a service (SaaS) applications, mobile applications and services, and any other computing applications and services. The system may include a first user 101, who may utilize a first user device 102 to access data, content, and applications, or to perform a variety of other tasks and functions. As an example, the first user 101 may utilize first user device 102 to access an application (e.g. a browser or a mobile application) executing on the first user device 102 that may be utilized to access web pages, data, and content associated with the system 100. In certain embodiments, the first user 101 may be any type of user that may desire to learn more about his existing health conditions, possible health conditions that may be likely in the future, personal abilities, activities that are suited for the first user 101, regimens suited for the first user 101, and/or any other information that may be utilized by the first user 101 to make enhanced decisions relating to his life. For example, the first user 101 may be an individual that is seeking to determine what health conditions that the first user 101 currently has, what health conditions the first user 101 is likely to have, and what regimen the first user 101 should deploy to reduce the likelihood of such health conditions from occurring. In certain embodiments, the first user 101 may be an individual that wants to learn more about any potential genetic anomalies that the first user 101 may have, to have the ability to predict potential health outcomes over time, and/or learn more about his genetic signature.

The first user device 102 utilized by the first user 101 may include a memory 103 that includes instructions, and a processor 104 that executes the instructions from the memory 103 to perform the various operations that are performed by the first user device 102. In certain embodiments, the processor 104 may be hardware, software, or a combination thereof. The first user device 102 may also include an interface 105 (e.g. screen, monitor, graphical user interface, audio device, neurotransmitter, etc.) that may enable the first user 101 to interact with various applications executing on the first user device 102, to interact with various applications executing within the system 100, and to interact with the system 100 itself. In certain embodiments, the first user device 102 may be a computer, a laptop, a tablet device, a phablet, a server, a mobile device, a smartphone, a smart watch, and/or any other type of computing device. Illustratively, the first user device 102 is shown as a mobile device in FIG. 1. The first user device 102 may also include a global positioning system (GPS), which may include a GPS receiver and any other necessary components for enabling GPS functionality, accelerometers, gyroscopes, sensors, and any other componentry suitable for a mobile device.

In addition to the first user 101, the system 100 may include a second user 110, who may utilize a second user device 111 to access data, content, and applications, or to perform a variety of other tasks and functions. As with the first user 101, the second user 110 may be a user that may desire to learn more about her existing health conditions, possible health conditions that may be likely in the future, personal abilities, activities that are suited for the second user 110, regimens suited for the second user 110, and/or any other information that may be utilized by the second user 110 to make enhanced decisions relating to her life. In certain embodiments, the second user 110 may be a physician whose patient is the first user 101, a fitness professional that trains and/or provides regimens to the first user 101, a psychologist and/or psychiatrist of the first user 101, a scientist that works with the first user 101, a dietitian that works with the first user 101, a caregiver of the first user 101, any type of individual that provides recommendations, training, decisions, and/or support for the first user 101. In certain embodiments, the first user 101 and/or any interactions conducted by the first user 101 with the system 100 may be configured to remain anonymous to the second user 110, other users, other systems, other programs, and/or other devices for a duration or indefinitely. In certain embodiments, the first user 101 and/or interactions conducted by the first user 101 with the system 100 may be identified and/or provided to the second user 110, such as if the second user 110 is a physician of the first user 101 or some other individual, device, and/or program with authorization.

Much like the first user 101, the second user 110 may utilize second user device 111 to access an application (e.g. a browser or a mobile application) executing on the second user device 111 that may be utilized to access web pages, data, and content associated with the system 100. The second user device 111 may include a memory 112 that includes instructions, and a processor 113 that executes the instructions from the memory 112 to perform the various operations that are performed by the second user device 111. In certain embodiments, the processor 113 may be hardware, software, or a combination thereof. The second user device 111 may also include an interface 114 (e.g. a screen, a monitor, a graphical user interface, etc.) that may enable the second user 110 to interact with various applications executing on the second user device 111, to interact with various applications executing in the system 100, and to interact with the system 100. In certain embodiments, the second user device 111 may be a computer, a laptop, a tablet device, a phablet, a server, a mobile device, a smartphone, a smart watch, and/or any other type of computing device. Illustratively, the second user device 111 may be a computing device in FIG. 1. The second user device 111 may also include any of the componentry described for first user device 102.

In certain embodiments, the first user device 102 and the second user device 111 may have any number of software applications and/or application services stored and/or accessible thereon. For example, the first and second user devices 102, 111 may include applications for determining and analyzing health conditions, applications for analyzing and determining genomic signatures, applications for determining health outcomes, applications for generating predictive models for predicting health outcomes and health progression, artificial intelligence applications, machine learning applications, big data applications, applications for analyzing data, applications for synthesizing data, applications for integrating data, cloud-based applications, search engine applications, natural language processing applications, database applications, algorithmic applications, phone-based applications, product-ordering applications, business applications, e-commerce applications, media streaming applications, content-based applications, database applications, gaming applications, internet-based applications, browser applications, mobile applications, service-based applications, productivity applications, video applications, music applications, social media applications, presentation applications, any other type of applications, any types of application services, or a combination thereof. In certain embodiments, the software applications and services may include one or more graphical user interfaces so as to enable the first and second users 101, 110 to readily interact with the software applications.

The software applications and services may also be utilized by the first and second users 101, 110 to interact with any device in the system 100, any network in the system 100, or any combination thereof. For example, the software applications executing on the first and second user devices 102, 111 may be applications for receiving data, applications for storing data, applications for determining health conditions, applications for determining activities that the first and/or second users 101, 110 are suited for, applications for determining regiments for the first and/or second users 101, 110, applications for receiving demographic and preference information, applications for transforming data, applications for executing mathematical algorithms, applications for generating and transmitting electronic messages, applications for generating and transmitting various types of content, any other type of applications, or a combination thereof. In certain embodiments, the first and second user devices 102, 111 may include associated telephone numbers, internet protocol addresses, device identities, or any other identifiers to uniquely identify the first and second user devices 102, 111 and/or the first and second users 101, 110. In certain embodiments, location information corresponding to the first and second user devices 102, 111 may be obtained based on the internet protocol addresses, by receiving a signal from the first and second user devices 102, 111, or based on profile information corresponding to the first and second user devices 102, 111.

The system 100 may also include a communications network 135. The communications network 135 of the system 100 may be configured to link each of the devices in the system 100 to one another. For example, the communications network 135 may be utilized by the first user device 102 to connect with other devices within or outside communications network 135. Additionally, the communications network 135 may be configured to transmit, generate, and receive any information and data traversing the system 100. In certain embodiments, the communications network 135 may include any number of servers, databases, or other componentry, and may be controlled by a service provider. The communications network 135 may also include and be connected to a cloud-computing network, a phone network, a wireless network, an Ethernet network, a satellite network, a broadband network, a cellular network, a private network, a cable network, the Internet, an internet protocol network, a content distribution network, any network, or any combination thereof. Illustratively, server 140 and server 150 are shown as being included within communications network 135.

Notably, the functionality of the system 100 may be supported and executed by using any combination of the servers 140, 150, and 160. The servers 140, and 150 may reside in communications network 135, however, in certain embodiments, the servers 140, 150 may reside outside communications network 135. The servers 140, and 150 may be utilized to perform the various operations and functions provided by the system 100, such as those requested by applications executing on the first and second user devices 102, 111. In certain embodiments, the server 140 may include a memory 141 that includes instructions, and a processor 142 that executes the instructions from the memory 141 to perform various operations that are performed by the server 140. The processor 142 may be hardware, software, or a combination thereof. Similarly, the server 150 may include a memory 151 that includes instructions, and a processor 152 that executes the instructions from the memory 151 to perform the various operations that are performed by the server 150. In certain embodiments, the servers 140, 150, and 160 may be network servers, routers, gateways, switches, media distribution hubs, signal transfer points, service control points, service switching points, firewalls, routers, edge devices, nodes, computers, mobile devices, or any other suitable computing device, or any combination thereof. In certain embodiments, the servers 140, 150 may be communicatively linked to the communications network 135, any network, any device in the system 100, or any combination thereof.

The database 155 of the system 100 may be utilized to store and relay information that traverses the system 100, cache information and/or content that traverses the system 100, store data about each of the devices in the system 100, and perform any other typical functions of a database. In certain embodiments, the database 155 may store the output from any operation performed by the system 100, operations performed and/or outputted by the artificial intelligence and machine learning system 206, operations performed and/or outputted by the electronic visualization tool 208, operations performed and/or outputted by any component, program, process, device, network of the system 100, or any combination thereof. For example, the database 155 may store data from data sources, such as, but not limited to, biochemistry data sources, physical measurement data sources, cognitive assessment data sources, genomics data sources, instrumentation measurement data sources, any type of data sources, or a combination thereof. In certain embodiments, the database 155 may be connected to or reside within the communications network 135, any other network, or a combination thereof. In certain embodiments, the database 155 may serve as a central repository for any information associated with any of the devices and information associated with the system 100. Furthermore, the database 155 may include a processor and memory or be connected to a processor and memory to perform the various operations associated with the database 155. In certain embodiments, the database 155 may be connected to the servers 140, 150, 160, the first user device 102, the second user device 111, the proprietary data warehouse 204, any devices in the system 100, any other device, any network, or any combination thereof.

The database 155 may also store information obtained from the system 100, store information associated with the first and second users 101, 110, store location information for the first and second user devices 102, 111 and/or first and second users 101, 110, store user profiles associated with the first and second users 101, 110, store device profiles associated with any device in the system 100, store communications traversing the system 100, store user preferences, store demographic information for the first and second users 101, 110, store information associated with any device or signal in the system 100, store information relating to usage of applications accessed by the first and second user devices 102, 111, store any information obtained from any of the networks in the system 100, store historical data associated with the first and second users 101, 110, store device characteristics, store information relating to any devices associated with the first and second users 101, 110, or any combination thereof. The database 155 may store algorithms for determining health conditions, algorithms for determining activities that the users are suited for, algorithms for determining abilities that the users have or can have, algorithms for the artificial intelligence and machine learning system 206, algorithms for determining relationships/matches between individual search criteria and health conditions, genomic information, and/or genetic anomalies, any other algorithms for performing any other calculations and/or operations in the system 100, or any combination thereof. In certain embodiments, the database 155 may be configured to store any information generated and/or processed by the system 100, store any of the information disclosed for any of the operations and functions disclosed for the system 100 herewith, store any information traversing the system 100, or any combination thereof. Furthermore, the database 155 may be configured to process queries sent to it by any device in the system 100.

The system 100 may also include a software application, which may be configured to perform and support the operative functions of the system 100. In certain embodiments, the application may be a website, a mobile application, a software application, or a combination thereof, which may be made accessible to users utilizing one or more computing devices, such as first user device 102 and second user device 111. The application of the system 100 may be accessible via an internet connection established with a browser program executing on the first or second user devices 102, 111, a mobile application executing on the first or second user devices 102, 111, or through other suitable means. Additionally, the application may allow users and computing devices to create accounts with the application and sign-in to the created accounts with authenticating username and password log-in combinations. The application may include a custom graphical user interface that the first user 101 or second user 110 may interact with by utilizing a web browser executing on the first user device 102 or second user device 111. In certain embodiments, the software application may execute directly as an installed program on the first and/or second user devices 102, 111.

The software application may include multiple programs and/or functions that execute within the software application and/or are accessible by the software application. For example, the software application may include an application that generates web content, pages, and/or data that may be accessible to the first and/or second user devices 102, 111, the proprietary data warehouse 204, the database 155 (e.g. reference databases), the electronic visualization tool 208 (e.g. web-based and/or other visualization tool), the artificial intelligence and machine learning systems 206, the external network 165, any type of program, any device and/or component of the system 100, or any combination thereof. The application that generates web content and pages may be configured to generate a graphical user interface and/or other types of interfaces for the software application that is accessible and viewable by the first and second users 101, 110 when the software application is loaded and executed on the first and/or second computing devices 102, 111. The graphical user interface for the software application (in certain embodiments, the electronic visualization tool 208) may display content associated with health conditions, measurement information taken by various types of instrumentation, genomics information, physical measurements, preventative action items, simulations of outbreaks, correlations of diseases and/or health conditions, information relating to population shifts in health and/or other areas, cognitive information, biochemistry information, health outcome information, predictive modeling information any other type of information, or any combination thereof. Additionally, the graphical user interface may display functionality provided by the software application that enables the first and/or second user 101, 110 and/or the first user device and/or second user device 111 to input parameters and requirements for the various process conducted by the system 100.

Referring now also to subsystem 200 of system 100, the system 100 may include an artificial intelligence and machine learning system 206, which may be comprised of hardware, software, or a combination thereof. The artificial intelligence and machine learning system 206 may include a series of modules and/or components for analyzing data and determining information relating to the data, such as the data obtained via the individual search criteria inputted into the system 100. Notably, the artificial intelligence and machine learning system 206 may include and incorporate the functionality of any existing artificial intelligence and machine learning system. In certain embodiments, the artificial intelligence and machine learning system 206 may include any necessary algorithms (e.g. mathematical and/or software algorithms) for supporting the functionality of the artificial intelligence and machine learning system 206. In certain embodiments, the artificial intelligence and machine learning system 206 may be configured to analyze individual search criteria and data contained in the database 155 (e.g. reference databases) to determine potential relationships and/or matches between the individual search criteria to one or more health conditions, genetic anomalies, diseases, any type of condition, or a combination thereof. The artificial intelligence and machine learning system 206 may also be configured to generate predictive models for determining health outcomes and progressions of diseases for an individual over time, such as by analyzing and synthesizing the data in the system 100. In further embodiments, the artificial intelligence and machine learning system 206 and the system 100 itself may conduct simulations for simulating outbreaks of health conditions, correlations of diseases, population shifts in health, preventative actions' effect on users, real-time monitoring of the users, or a combination thereof.

The system 100 may also include any number of proprietary data warehouses 204. The proprietary data warehouses 204 may be databases and/or data warehouses that may be utilized to aggregate and store historical individual search criteria in a format suitable for analysis of the data by the internal and/or external components (e.g. external network 165) of the system 100. In certain embodiments, the system 100 may be configured to update and aggregate data and information from the proprietary data warehouses 204 with the individual search criteria inputted into the system 100 and/or metadata (i.e. information describing and/or related to the individual search criteria) associated with the individual search criteria. In certain embodiments, the databases 155 and/or proprietary data warehouses 204 may be configured to store health condition information, findings and/or analyses generated by the system 100, or a combination thereof. In certain embodiments, the proprietary data warehouses 204 may include a history of all cases, users, and/or associated data that are in and/or made accessible to the system 100. The system 100 may further include an electronic visualization tool 208, which may be configured to generate media content, such as, but not limited to, audio content, video content, graph content, analysis content, web-based content, sensory content, haptic content, any type of content, which may be visualized and/or heard via an application supporting the functionality of the system 100. The electronic visualization tool 208 may comprise software, hardware, or both, and may include any number of processors and/or memories to support its functionality. The electronic visualization tool 208 may also render any of the data and/or information traversing the system 100, such as, but not limited to, the individual search criteria, health conditions, health outcomes, predictive model information, preventative action information, aggregated research information, simulation information relating to simulations conducted in the system 100, monitoring information associated with monitoring users of the system 100, any other information, or a combination thereof. In certain embodiments, information generated by the electronic visualization tool 208 may be provided as genetic decision support feedback for health professionals and/or others to further processing and/or review. In certain embodiments, the electronic visualization tool 208 may be web-based, application-based, device-based, or a combination thereof. In certain embodiments, the electronic visualization tool 208 may be configured to conduct and executed simulations based on the aggregated data and/or other data of the system 100, conduct research, or a conduct a combination thereof.

The system 100 may also include an external network 165. The external network 165 of the system 100 may be configured to link each of the devices in the system 100 to one another. For example, the external network 165 may be utilized by the first user device 102 to connect with other devices within or outside communications network 135. Additionally, the external network 165 may be configured to transmit, generate, and receive any information and data traversing the system 100. In certain embodiments, the external network 165 may include any number of servers, databases, or other componentry, and may be controlled by a service provider. The external network 165 may also include and be connected to a cloud-computing network, a phone network, a wireless network, an Ethernet network, a satellite network, a broadband network, a cellular network, a private network, a cable network, the Internet, an internet protocol network, a content distribution network, any network, or any combination thereof. In certain embodiments, the external network 165 may be outside the system 100 and may be configured to perform various functionality provided by the system 100, such as if the system 100 is overloaded and/or needs additional processing resources.

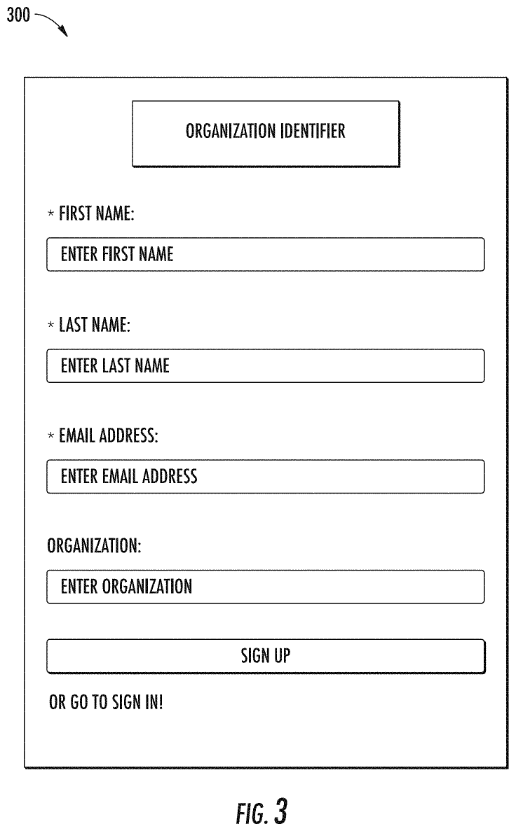

Operatively and referring now also to FIGS. 3-9, the system 100 may operate according to the following exemplary use-case scenarios. Notably, the system 100 is not limited to the specific use-case scenarios described herein, and may be applied to any suitable and/or desired use-case scenario. In a first use-case scenario, a user, such as first user 101, may access an application supporting the functionality of the system 100, such as by utilizing first user device 102. Upon accessing the application, a graphical user interface of the application may be rendered by using the electronic visualization tool 208. As an example, if it is the first time that the first user 101 is utilizing the application of the system 100, the graphical user interface may display screen 300, as shown in FIG. 3. The screen 300 may be configured to receive inputs from the first user 101, which may be utilized to create a unique and private account that will enable the first user 101 to access the system 100 again on subsequent occasions. The screen 300 may be configured to take any type of inputs from the first user 101 and may be configured to include any desired fields. In certain embodiments, the screen 300 may include an organization identifier that identifies an organization associated with the application and/or system 100, an input field for a first name of the first user 101, an input field for a last name of the first user 101, an input field for an email address of the first user 101, an organization field for an organization associated with the first user 101, and a sign-up digital button, which, when selected, causes the account for the first user 101 to be created in the system 100. In certain embodiments, the first user 101 may be prompted to enter in a desired username and/or password combination so that the first user 101 can log into the application on subsequent occasions.

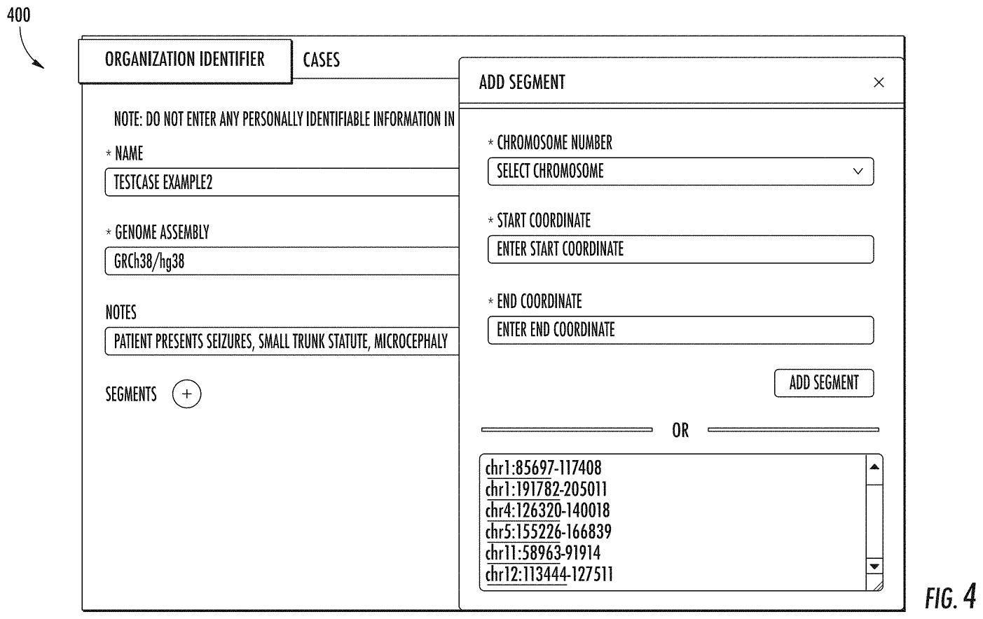

Once the account has been created for the first user 101, the application may enable the first user 101 to input any desired individual search criteria as inputs into the system 100 for search and analysis by the system 100. As described elsewhere in the present disclosure, the individual search criteria may include, but is not limited to, keywords, genomic signature information, phenotype information, saliva information, blood information, information obtained from medical devices, any physiological information, any medical information, lifestyle information associated with the first user 101, anatomic information, neurotransmitter information, information obtained via microphones, biochemical information, DNA information, medical history information, video content, audio content, sensory content, haptic content, and/or other information associated with the first user 101. Illustratively, and as an example, screen 400 of FIG. 4 enables the first user 101 to enter in individual search criteria corresponding to genomic chromosome segments, such as the genomic chromosome segments of the first user 101 himself Screen 400 allows the first user 101 to enter in individual search criteria, such as, but not limited to, the first user's 101 chromosome number, a start coordinate for the chromosome, and an end coordinate for the chromosome. Additionally, in certain embodiments, the screen 400 may allow the first user 101 to enter in individual segments of the genomic chromosome, as is shown at the bottom input box of screen 400. In certain embodiments, the first user 101 may add any number of additional segments, such as by selecting the add segment digital button of screen 400.

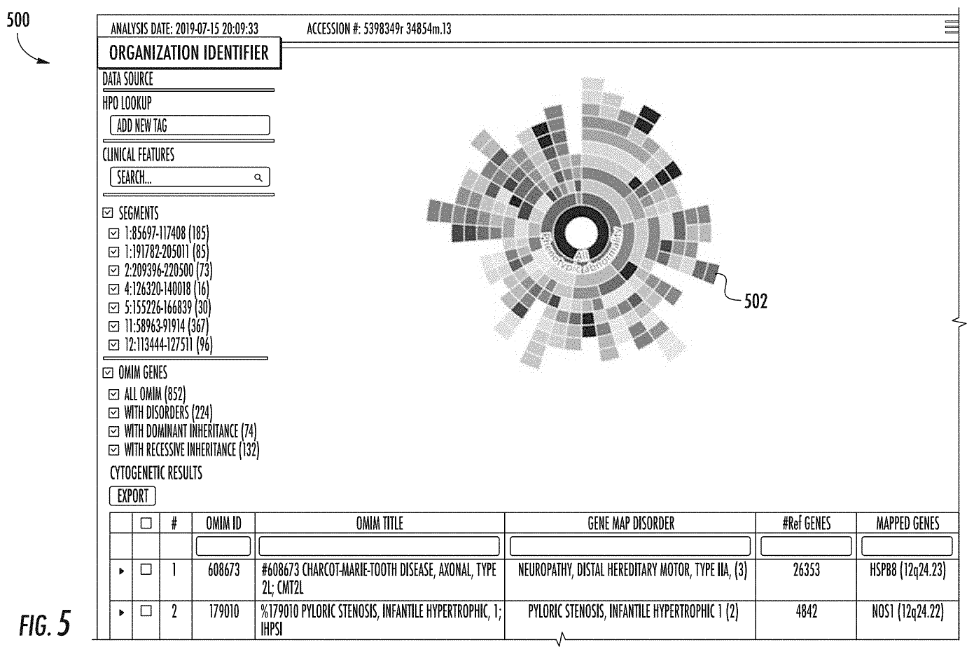

Once the individual search criteria are entered into the screen 400, the system 100 may convert and format the received individual search criteria into a format suitable for communication, storage, synthesis, and analysis by components of the system 100. The system 100 may query one or more reference databases 155 and/or download relevant health data for conducting an analysis based on the individual search criteria. The system 100 may compare the individual search criteria to the data and contents of the reference databases 155 (such as by utilizing mathematical algorithms) to determine potential relationships and/or matches between the individual search criteria and the data and contents found in the reference databases 155. Based on the comparing and referring now also to FIG. 5, the system 100 may generate, such as via the electronic visualization tool 208, a screen 500 that visualizes results derived from the comparing and analysis conducted by the artificial intelligence and machine learning system components (e.g. artificial intelligence and machine learning system 206) of the system 100 on the individual search criteria. In screen 500, the system 100 may generate, such as via the electronic visualization tool 208 a sunburst image that visualizes the data and potential relationships and/or matches between the individual search criteria and the contents of the reference databases 155. For example, in FIG. 5, the sunburst illustrates all phenotypic abnormalities detected by the system 100 for all of the chromosome segments entered in the individual search criteria in comparison to references database 155, such as Online Mendelian Inheritance in Man (OMIM) Genes (e.g. all OMIM including disorders, dominant inheritance, and/or recessive inheritance). In certain embodiments, each block 502 in the sunburst may be clickable and details of each block 502 may be displayed on the screen 500. In certain embodiments, the OMIM ID, the OMIM title, the detected gene map disorder, the reference gene(s), and/or the mapped gene(s) may be visualized and identified and displayed on screen 500, such as in a table. In certain embodiments, the first user 101 may click on each row of the table to learn more about each result. For example, if the first user 101 clicks on the first row generated, the system 100 may automatically provide additional information related to the row, such as a description of neuropathy, conditions associated with neuropathy, segments associated with the neuropathy, treatments for neuropathy, preventive actions for avoiding or combating neuropathy, any other information, or a combination thereof.

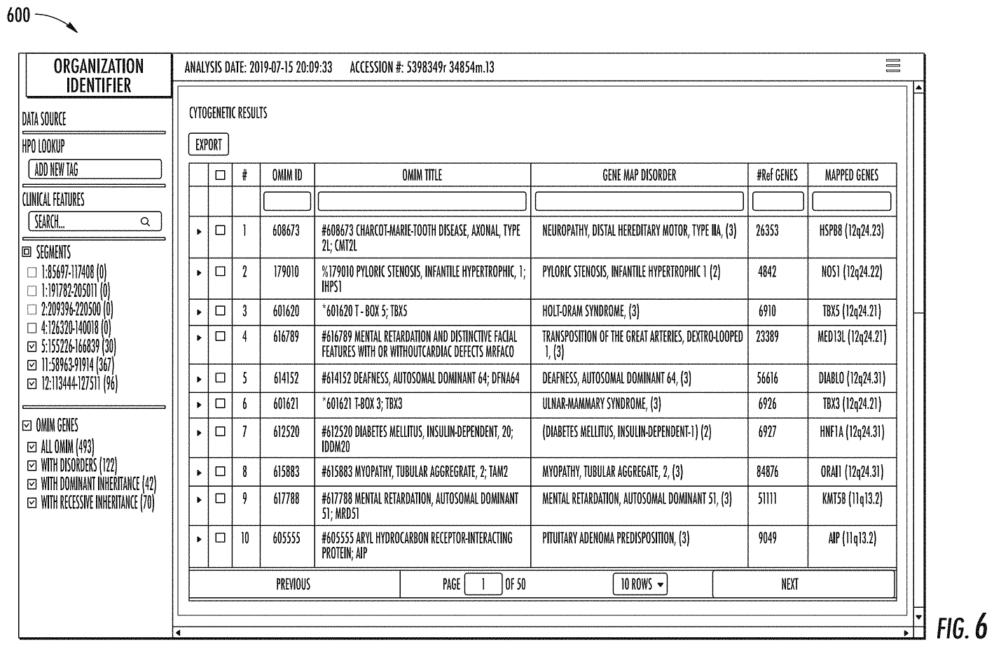

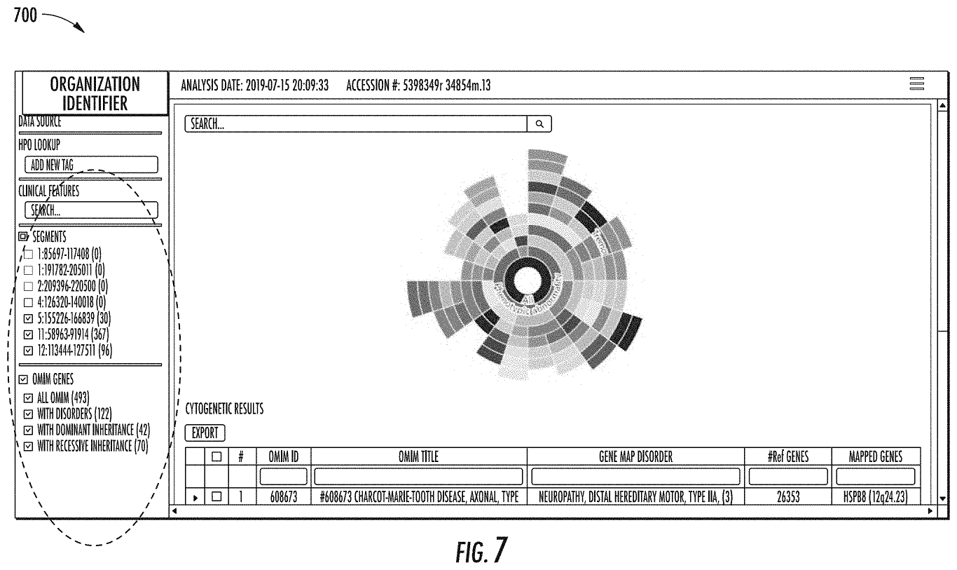

In certain embodiments, certain individual search criteria may be filtered out so that an individual and/or program and/or device analyzing the results may perform further more details analyses so as to identify which segments are of greatest interest, such as which segments are associated with the most severe and/or serious disorders and/or health conditions. As shown in FIG. 6, the first user 101 may deselect some of the segments so that the system 100 conducts a further comparison to the reference databases based on a subset of the segments. For example, in screen 600 of FIG. 6 the first four segments have been deselected and the remaining three segments remain selected. Upon deselecting each of the segments, the system 100 may automatically perform the comparison to the reference database 155 without further intervention by the first user 101. In other words, as the first user 101 deselects search criteria (or otherwise adjusts the search criteria), the data shown in the table in screen 600 (or screen 500) may automatically update in real-time (e.g. such as via a real-time simulation) to show the appropriate results for the current individual search criteria. For example, the table in screen 600 automatically updates the results displayed in screen 600 as the first user 101 deselects each of the first four chromosome segments. Additionally, in addition to updating the rows of the table shown in screen 600, the sunburst may also be updated (such as via a real-time simulation) based on the new individual search criteria. For example and referring now also to screen 700 of FIG. 7, the sunburst has been updated now that the first four chromosome segments are no longer selected for the individual search criteria. Now, the sunburst visualizes the data and relationships between the search criteria and the reference databases 155 for the remaining three chromosome segments.

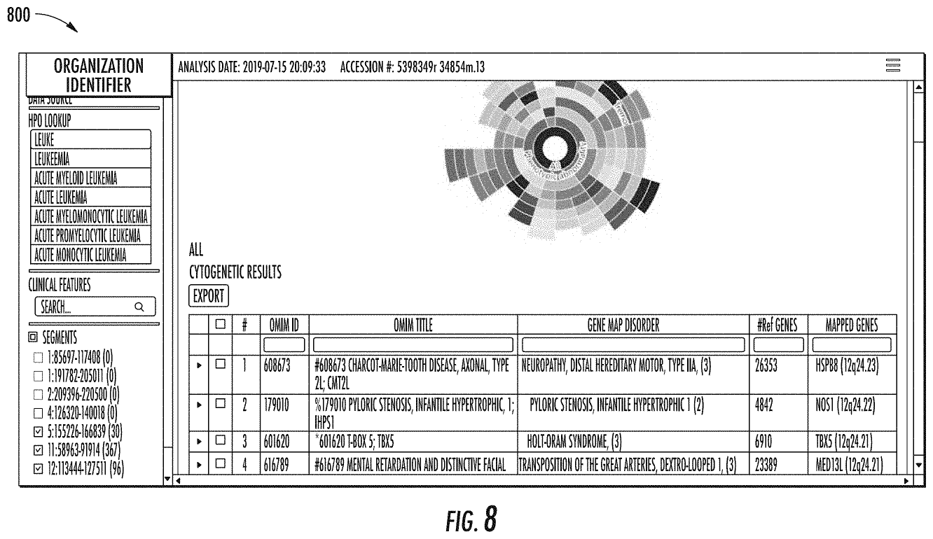

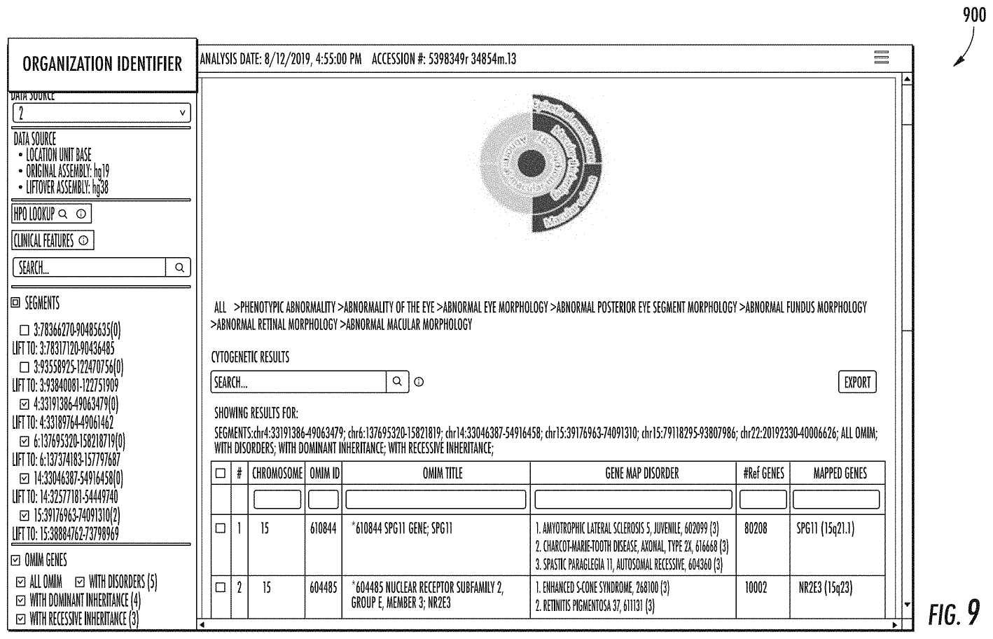

In addition to adjusting individual search criteria by adjusting the specific chromosome segments that the first user 101 wants to analyze via the system 100, the first user 101 may also adjust the individual search criteria by entering in additional keywords (or other types of search criteria) into a search function (e.g. lookup function) of the application. For example and referring now also to screen 800 of FIG. 8, the first user 101 may start entering in leukemia into the search field and, as the first user 101 is entering in the search term, the system 100 may also suggest other search criteria associated with leukemia that may be of interest to the first user 101. If the first user 101 selects leukemia or a suggested other search criteria, the system 100 may regenerate the sunburst and may update the table including the table entries including the detected genetic disorders and/or health conditions in real-time. Notably, the first user 101 may adjust the individual search criteria as the first user 101 desires, and all data may be updated in real-time for the first user 101 to allow for rapid and efficient access to the results. In certain embodiments, the first user 101 may select (e.g. by selecting via a mouse or other input mechanism such as a keyboard) one of the blocks 502 to conduct further research. If the user selects one of the blocks 502 associated with detected eye abnormalities, the system 100 may generate the sub-sunburst visualized in screen 900 of FIG. 9. In this case, the sub-sunburst has its own blocks 502 and the blocks 502 are directed to abnormal macular morphology, macular thickening, epiretinal membrane, and macular edema, which are all associated with detected eye disorders and/or conditions. In certain embodiments, the center of the sunburst may be for a general detected condition and/or disorder and the blocks 502 layered outwards from the center may progressively be more specific features and/or characteristics associated with the detected condition and/or disorder. Notably, the system 100 may be utilized for any type of search criteria and/or analyses and may be combined with the methods described herein.

In certain embodiments, the system 100 may be utilized with any other desired use-case scenario. For example, in one use-case scenario, the system 100 may be utilized in the context of the pharmaceutical industry. The scenario may involve analyzing search criteria associated with a specific syndrome and/or condition, such as cystic fibrosis. In this use-case scenario drug development research may be conducted where a predicted link to a genetic marker for a new drug does not align with the genetic variation and/or anticipated efficacy. Additional genetic markers may be utilized statistically to address underfitting as a result of non-linear associations, and overall drug efficacy may seemingly be a random departure from known genetic variation, even though each genetic marker protein appears to be associated with the syndrome. The system 100 may be utilized in such a context. In particular, individual search criteria may be inputted into the system 100 and symptoms of the disorder may be utilized as search parameters for unknown genetic markers. Exemplary searches may be as follows: Search 1: Search for similar clinical features as those characterized by a syndrome (e.g. cystic fibrosis). Look for other genetic markers sharing commonality with the syndrome. Search 1a: Search subsets and varying combinations of clinical features with a specific syndrome and look for alternate common genetic markers or pathways. Search 2: Reverse the search and then utilize markers revealed by original searches to identify related syndromes. Search 3: Utilize the historic data contained in the proprietary data warehouses 204 to simulate potential prevalence of a condition therefore estimating the potential market demand of a new drug. By using the system 100, the user may have the ability to view and analyze clinical features as a window to genetic similarities and variants. Additionally, the user may have the ability to search revealed sets and subset to characterize genetic commonalities, and the user and/or system 100 itself may have the ability to learn from the search results to provide and generate new search parameters for further analyses to be conducted by the system 100.

As another use-case scenario, the system 100 may be utilized in the context of a health system. As an example, the use-case scenario may be related to breast cancer. Notably, BRCA1 and BRCA2 account for 70% or less of the overall genetic variation associated with the disease. Therefore, 30% of the time women with the genes are not going to develop breast cancer. Systematically adding family history increases the percentage to more than 90%. In this context, the system 100 functionality may be invaluable in: a) identifying the associated genetic variants that a drug can target; and b) extending the overall ability to predict and propose prophylactic treatment. For example, by using the system 100, individual data samples with high dimensionality can be used to explore disease states and genetic profiles for women that are or are not characterized by expected genetic variants. The system 100 may also provide the ability to use individual data to determine the highest-value predictive pathways. As a further us-case scenario, the system 100 may be utilized in the context of a laboratory. In this use-case scenario, research facilities will be able to “drill” into genetic data using the associated health information to navigate, examine, and characterize genetic commonalities. Notably, tumors often begin with a mutation that causes the cell to overgrow its barriers. However, the increased growth causes the cell to subsequently mutate. The question then becomes which mutation was first and is the first mutation the one that should be used to guide drug development and/or therapy? By using the outputs and functionality of the system 100, patients' histories and genetic data in “cis” (concomitant) will provide more definitive associations between mutations and symptoms that determine the “potent” mutations.

Notably, as shown in FIG. 1, the system 100 may perform any of the operative functions disclosed herein by utilizing the processing capabilities of server 160, the storage capacity of the database 155, or any other component of the system 100 to perform the operative functions disclosed herein. The server 160 may include one or more processors 162 that may be configured to process any of the various functions of the system 100. The processors 162 may be software, hardware, or a combination of hardware and software. Additionally, the server 160 may also include a memory 161, which stores instructions that the processors 162 may execute to perform various operations of the system 100. For example, the server 160 may assist in processing loads handled by the various devices in the system 100, such as, but not limited to, receiving the individual search criteria, generating digital files including the individual search criteria, converting the digital into formats useable for communication, storage, synthesis, and/or analysis by components of the system 100, comparing the search criteria to contents of the reference databases by utilizing mathematical algorithms; determining potential relationships and/or matches between search criteria and information in the reference databases; visualizing the information determined and analyzed by the system 100 via an electronic visualization tool, resetting individual search criteria, aggregating historical individual search criteria and information in a format suitable for analysis by the system 100; formatting data for future re-use by the system in additional system data analysis and by-products; automatically updating and/or aggregating the proprietary data warehouse 204 with the individual search criteria and metadata associated with the individual search criteria; and performing any other suitable operations conducted in the system 100 or otherwise. In one embodiment, multiple servers 160 may be utilized to process the functions of the system 100. The server 160 and other devices in the system 100, may utilize the database 155 for storing data about the devices in the system 100 or any other information that is associated with the system 100. In one embodiment, multiple databases 155 may be utilized to store data in the system 100.

Although FIGS. 1-3 illustrates specific example configurations of the various components of the system 100, the system 100 may include any configuration of the components, which may include using a greater or lesser number of the components. For example, the system 100 is illustratively shown as including a first user device 102, a second user device 111, a database 125, a communications network 135, a server 140, a server 150, a server 160, a database 155, an external network 165, an artificial intelligence and machine learning system 206, an electronic visualization tool 208, unique case information 202 associated with a user (including phenotype information, genetic anomaly information, etc.), and proprietary data warehouses 204. However, the system 100 may include multiple first user devices 102, multiple second user devices 111, multiple databases 125, multiple communications networks 135, multiple servers 140, multiple servers 150, multiple servers 160, multiple databases 155, multiple data warehouses 204, multiple artificial intelligence and machine learning systems 206, multiple unique case information 202, multiple electronic visualization tools 208, multiple external networks 165, and/or any number of any of the other components inside or outside the system 100. Similarly, the system 100 may include any number of data sources, applications, systems, and/or programs. Furthermore, in certain embodiments, substantial portions of the functionality and operations of the system 100 may be performed by other networks and systems that may be connected to system 100.

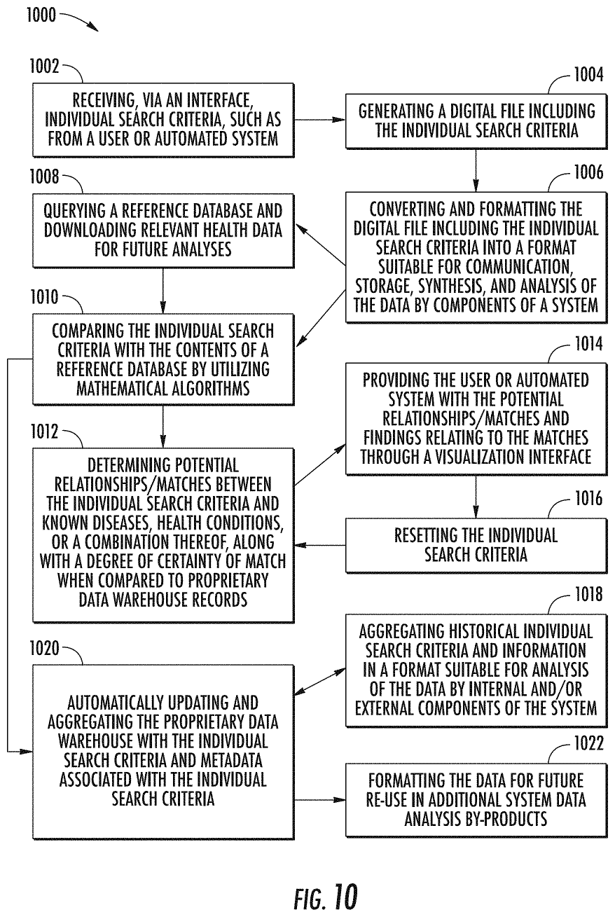

As shown in FIG. 10, an exemplary method 1000 for providing dynamic, real-time, genomics decision support and simulation the use of machine learning and other techniques and processes is schematically illustrated. The method 1000 may include, at step 1002, receiving individual search criteria associated with an individual (e.g. first user 101) from one or more sources of a plurality of sources of data. For example, the search criteria may be received from first user device 102 from first user 101 and may include keywords, genomic signature information, phenotype information, saliva information, blood information, information obtained from medical devices (e.g. MM scans, PET scans, CT scans, thermometer readings, blood pressure readings, heart rate readings, stress readings, echocardiograms, etc.), any physiological information, any medical information, lifestyle information associated with the first user 101, anatomic information, neurotransmitter information, information obtained via microphones, biochemical information, DNA information, medical hi story information, video content, audio content (e.g. voice content, etc.), sensory content, haptic content, and/or other information associated with the first user 101. In certain embodiments, the receiving of the individual search criteria may be performed and/or facilitated by utilizing the first user device 102, the second user device 111, the server 140, the server 150, the server 160, the communications network 136, the external network 165, the database 155, the proprietary data warehouses 204, the artificial intelligence and machine learning system 206, any appropriate program, device, network, and/or process of the system 100, or a combination thereof. At step 1004, the method 1000 may include generating a digital file including the individual search criteria. In certain embodiments, the generating may be performed and/or facilitated by utilizing the first user device 102, the second user device 111, the server 140, the server 150, the server 160, the communications network 136, the external network 165, the database 155, the proprietary data warehouses 204, the artificial intelligence and machine learning system 206, any appropriate program, device, network, and/or process of the system 100, or a combination thereof.

At step 1006, the method 1000 may include converting and/or formatting the digital file including the individual search criteria into a format suitable for communication, synthesis, storage, and/or analysis of the data included in the individual search criteria by components of the system 100. In certain embodiments, the converting and/or formatting may be performed and/or facilitated by utilizing the first user device 102, the second user device 111, the server 140, the server 150, the server 160, the communications network 136, the external network 165, the database 155, the proprietary data warehouses 204, the artificial intelligence and machine learning system 206, any appropriate program, device, network, and/or process of the system 100, or a combination thereof. At step 1008, the method 1000 may include querying a reference database and downloading relevant health data for analyses to be conducted by the system 100. In certain embodiments, the querying may be performed and/or facilitated by utilizing the first user device 102, the second user device 111, the server 140, the server 150, the server 160, the communications network 136, the external network 165, the database 155, the proprietary data warehouses 204, the artificial intelligence and machine learning system 206, any appropriate program, device, network, and/or process of the system 100, or a combination thereof.

At step 1010, the method 1000 may include comparing the individual search criteria with contents of the reference database by utilizing mathematical algorithms. In certain embodiments, the comparing may be performed and/or facilitated by utilizing the first user device 102, the second user device 111, the server 140, the server 150, the server 160, the communications network 136, the external network 165, the database 155, the proprietary data warehouses 204, the artificial intelligence and machine learning system 206, any appropriate program, device, network, and/or process of the system 100, or a combination thereof. At step 1012, the method 1000 may include determining potential relationships and/or potential matches between the individual search criteria and known diseases, health conditions, or a combination thereof, along with a degree of certainty of the relationship and/or match when compared to records contained in one or more proprietary data warehouses 204. In certain embodiments, the determining may be performed and/or facilitated by utilizing the first user device 102, the second user device 111, the server 140, the server 150, the server 160, the communications network 136, the external network 165, the database 155, the proprietary data warehouses 204, the artificial intelligence and machine learning system 206, any appropriate program, device, network, and/or process of the system 100, or a combination thereof.

At step 1014, the method 1000 may include providing the user (e.g. first user 101) and/or an automated system with the determined potential relationships and/or matches and findings relating to the relationships and/or matches through a visualization interface, such as an electronic visualization tool 208. In certain embodiments, the providing may be performed and/or facilitated by utilizing the first user device 102, the second user device 111, the server 140, the server 150, the server 160, the communications network 136, the external network 165, the database 155, the proprietary data warehouses 204, the artificial intelligence and machine learning system 206, any appropriate program, device, network, and/or process of the system 100, or a combination thereof. At step 1016, the method 1000 may include resetting the individual search criteria so that new inputs may be inputted into the system 100 for further training the system 100, such as the artificial intelligence and machine learning system 206 of the system 100. In certain embodiments, the resetting of the search criteria may be performed and/or facilitated by utilizing the first user device 102, the second user device 111, the server 140, the server 150, the server 160, the communications network 136, the external network 165, the database 155, the proprietary data warehouses 204, the artificial intelligence and machine learning system 206, any appropriate program, device, network, and/or process of the system 100, or a combination thereof.

At step 1018, the method 1000 may include aggregating historical individual search criteria and information in a format suitable for analysis of the data by the internal and/or external components of the system 100. In certain embodiments, the aggregating may be performed and/or facilitated by utilizing the first user device 102, the second user device 111, the server 140, the server 150, the server 160, the communications network 136, the external network 165, the database 155, the proprietary data warehouses 204, the artificial intelligence and machine learning system 206, any appropriate program, device, network, and/or process of the system 100, or a combination thereof. At step 1020, the method 1000 may include automatically updating and aggregating the data in the proprietary data warehouses 204 of the system 100 with the individual search criteria and metadata associated with the individual search criteria. In certain embodiments, the updating and/or aggregating may be performed and/or facilitated by utilizing the first user device 102, the second user device 111, the server 140, the server 150, the server 160, the communications network 136, the external network 165, the database 155, the proprietary data warehouses 204, the artificial intelligence and machine learning system 206, any appropriate program, device, network, and/or process of the system 100, or a combination thereof. At step 1022, the method 1000 may include formatting the data in the proprietary data warehouses 204 (and/or elsewhere in the system 100) for future re-use in additional system data analysis by-products. In certain embodiments, the method 1000 may include conducting real-time monitoring of the individual (e.g. first user 101) associated with the individual search criteria, generating predictive models for predicting health outcomes and/or health progression in an individual, determining preventative actions for reversing and/or preventing health outcomes and/or existing health conditions, aggregating research data, conducting simulations of outbreaks, generating and determining correlations between various diseases and/or health conditions based on the analyses conducted in the system 100, and/or determining shifts in health in various populations. Notably, the method 1000 may further incorporate any of the features and functionality described for the system 100 or as otherwise described herein.

The systems and methods disclosed herein may include additional functionality and features. For example, the operative functions of the system 100 and method may be configured to execute on a special-purpose processor specifically configured to carry out the operations provided by the system 100 and method. Notably, the operative features and functionality provided by the system 100 and method may increase the efficiency of computing devices that are being utilized to facilitate the functionality provided by the system 100 and method 1000. For example, through the use of the artificial intelligence and machine learning system 206, a reduced amount of computer operations need to be performed by the devices in the system 100 using the processors and memories of the system 100 than in systems that are not capable of machine learning as described in this disclosure. In such a context, less processing power needs to be utilized because the processors and memories do not need perform analyses and operations that have already been learned by the system 100. As a result, there are substantial savings in the usage of computer resources by utilizing the software, functionality, and algorithms provided in the present disclosure.

Notably, in certain embodiments, various functions and features of the system 100 and methods may operate without human intervention and may be conducted entirely by computing devices, robots, and/or processes. For example, in certain embodiments, multiple computing devices may interact with devices of the system 100 to provide the functionality supported by the system 100. Additionally, in certain embodiments, the computing devices of the system 100 may operate continuously to reduce the possibility of errors being introduced into the system 100. In certain embodiments, the system 100 and methods may also provide effective computing resource management by utilizing the features and functions described in the present disclosure. For example, in certain embodiments, while determining potential relationships and/or matches associated with an individual (and/or any other information that may be of use to the individual) based on search criteria and information obtained from reference databases 155 and/or proprietary data warehouses 204, any selected device in the system 100 may transmit a signal to a computing device receiving or processing the input that only a specific quantity of computer processor resources (e.g. processor clock cycles, processor speed, processor cache, etc.) may be dedicated to processing the data utilized to determine the potential relationship and/or match, any other operation conducted by the system 100, or any combination thereof. For example, the signal may indicate an amount of processor cycles of a processor that may be utilized to process the data, and/or specify a selected amount of processing power that may be dedicated to processing the data or any of the operations performed by the system 100. In certain embodiments, a signal indicating the specific amount of computer processor resources or computer memory resources to be utilized for performing an operation of the system 100 may be transmitted from the first and/or second user devices 102, 111 to the various components and devices of the system 100.

In certain embodiments, any device in the system 100 may transmit a signal to a memory device to cause the memory device to only dedicate a selected amount of memory resources to the various operations of the system 100. In certain embodiments, the system 100 and methods may also include transmitting signals to processors and memories to only perform the operative functions of the system 100 and methods at time periods when usage of processing resources and/or memory resources in the system 100 is at a selected, predetermined, and/or threshold value. In certain embodiments, the system 100 and methods may include transmitting signals to the memory devices utilized in the system 100, which indicate which specific portions (e.g. memory sectors, etc.) of the memory should be utilized to store any of the data utilized or generated by the system 100. Notably, the signals transmitted to the processors and memories may be utilized to optimize the usage of computing resources while executing the operations conducted by the system 100. As a result, such features provide substantial operational efficiencies and improvements over existing technologies.

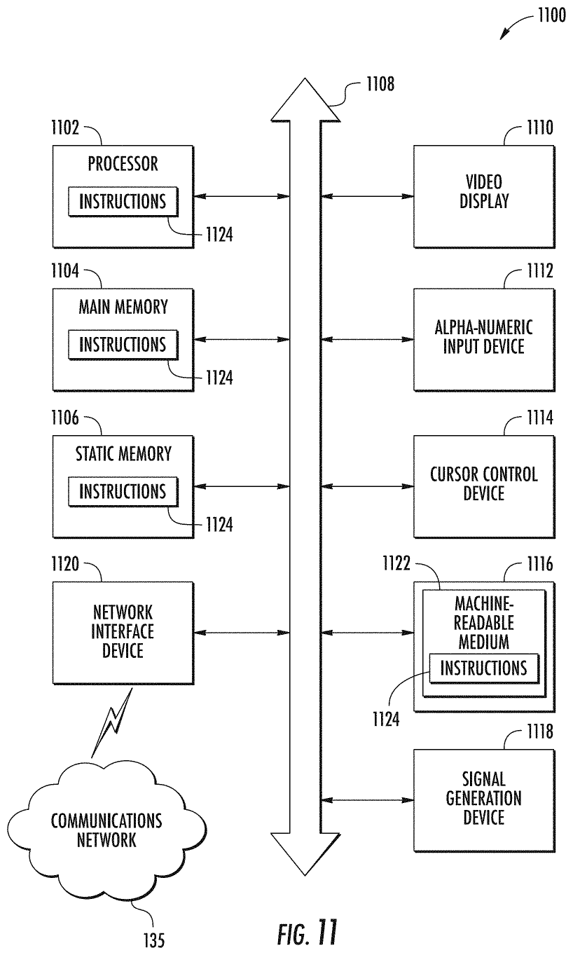

Referring now also to FIG. 11, at least a portion of the methodologies and techniques described with respect to the exemplary embodiments of the system 100 can incorporate a machine, such as, but not limited to, computer system 1100, or other computing device within which a set of instructions, when executed, may cause the machine to perform any one or more of the methodologies or functions discussed above. The machine may be configured to facilitate various operations conducted by the system 100. For example, the machine may be configured to, but is not limited to, assist the system 100 by providing processing power to assist with processing loads experienced in the system 100, by providing storage capacity for storing instructions or data traversing the system 100, or by assisting with any other operations conducted by or within the system 100.

In some embodiments, the machine may operate as a standalone device. In some embodiments, the machine may be connected (e.g., using communications network 135, another network, or a combination thereof) to and assist with operations performed by other machines, programs, functions, and systems, such as, but not limited to, the first user device 102, the second user device 111, the server 140, the server 150, the database 155, the server 160, the artificial intelligence and machine learning system 204, the electronic visualization tool 208, the external network 165, the communications network 135, any device, system, and/or program in FIGS. 1-11, or any combination thereof. The machine may be connected with any component in the system 100. In a networked deployment, the machine may operate in the capacity of a server or a client user machine in a server-client user network environment, or as a peer machine in a peer-to-peer (or distributed) network environment. The machine may comprise a server computer, a client user computer, a personal computer (PC), a tablet PC, a laptop computer, a desktop computer, a control system, a network router, switch or bridge, or any machine capable of executing a set of instructions (sequential or otherwise) that specify actions to be taken by that machine. Further, while a single machine is illustrated, the term “machine” shall also be taken to include any collection of machines that individually or jointly execute a set (or multiple sets) of instructions to perform any one or more of the methodologies discussed herein.

The computer system 1100 may include a processor 1102 (e.g., a central processing unit (CPU), a graphics processing unit (GPU, or both), a main memory 1104 and a static memory 1106, which communicate with each other via a bus 1108. The computer system 1100 may further include a video display unit 1110, which may be, but is not limited to, a liquid crystal display (LCD), a flat panel, a solid state display, or a cathode ray tube (CRT). The computer system 1100 may include an input device 1112, such as, but not limited to, a keyboard, a cursor control device 1114, such as, but not limited to, a mouse, a disk drive unit 1116, a signal generation device 1118, such as, but not limited to, a speaker or remote control, and a network interface device 1120.

The disk drive unit 1116 may include a machine-readable medium 1122 on which is stored one or more sets of instructions 1124, such as, but not limited to, software embodying any one or more of the methodologies or functions described herein, including those methods illustrated above. The instructions 1124 may also reside, completely or at least partially, within the main memory 1104, the static memory 1106, or within the processor 1102, or a combination thereof, during execution thereof by the computer system 1100. The main memory 1104 and the processor 1102 also may constitute machine-readable media.

Dedicated hardware implementations including, but not limited to, application specific integrated circuits, programmable logic arrays and other hardware devices can likewise be constructed to implement the methods described herein. Applications that may include the apparatus and systems of various embodiments broadly include a variety of electronic and computer systems. Some embodiments implement functions in two or more specific interconnected hardware modules or devices with related control and data signals communicated between and through the modules, or as portions of an application-specific integrated circuit. Thus, the example system is applicable to software, firmware, and hardware implementations.

In accordance with various embodiments of the present disclosure, the methods described herein are intended for operation as software programs running on a computer processor. Furthermore, software implementations can include, but not limited to, distributed processing or component/object distributed processing, parallel processing, or virtual machine processing can also be constructed to implement the methods described herein.

The present disclosure contemplates a machine-readable medium 1122 containing instructions 1124 so that a device connected to the communications network 135, the external network 165, another network, or a combination thereof, can send or receive voice, video or data, and communicate over the communications network 135, the external network 165, another network, or a combination thereof, using the instructions. The instructions 1124 may further be transmitted or received over the communications network 135, the external network 165, another network, or a combination thereof, via the network interface device 1120.

While the machine-readable medium 1122 is shown in an example embodiment to be a single medium, the term “machine-readable medium” should be taken to include a single medium or multiple media (e.g., a centralized or distributed database, and/or associated caches and servers) that store the one or more sets of instructions. The term “machine-readable medium” shall also be taken to include any medium that is capable of storing, encoding or carrying a set of instructions for execution by the machine and that causes the machine to perform any one or more of the methodologies of the present disclosure.

The terms “machine-readable medium,” “machine-readable device,” or “computer-readable device” shall accordingly be taken to include, but not be limited to: memory devices, solid-state memories such as a memory card or other package that houses one or more read-only (non-volatile) memories, random access memories, or other re-writable (volatile) memories; magneto-optical or optical medium such as a disk or tape; or other self-contained information archive or set of archives is considered a distribution medium equivalent to a tangible storage medium. The “machine-readable medium,” “machine-readable device,” or “computer-readable device” may be non-transitory, and, in certain embodiments, may not include a wave or signal per se. Accordingly, the disclosure is considered to include any one or more of a machine-readable medium or a distribution medium, as listed herein and including art-recognized equivalents and successor media, in which the software implementations herein are stored.

The illustrations of arrangements described herein are intended to provide a general understanding of the structure of various embodiments, and they are not intended to serve as a complete description of all the elements and features of apparatus and systems that might make use of the structures described herein. Other arrangements may be utilized and derived therefrom, such that structural and logical substitutions and changes may be made without departing from the scope of this disclosure. Figures are also merely representational and may not be drawn to scale. Certain proportions thereof may be exaggerated, while others may be minimized. Accordingly, the specification and drawings are to be regarded in an illustrative rather than a restrictive sense.

Thus, although specific arrangements have been illustrated and described herein, it should be appreciated that any arrangement calculated to achieve the same purpose may be substituted for the specific arrangement shown. This disclosure is intended to cover any and all adaptations or variations of various embodiments and arrangements of the invention. Combinations of the above arrangements, and other arrangements not specifically described herein, will be apparent to those of skill in the art upon reviewing the above description. Therefore, it is intended that the disclosure not be limited to the particular arrangement(s) disclosed as the best mode contemplated for carrying out this invention, but that the invention will include all embodiments and arrangements falling within the scope of the appended claims.

The foregoing is provided for purposes of illustrating, explaining, and describing embodiments of this invention. Modifications and adaptations to these embodiments will be apparent to those skilled in the art and may be made without departing from the scope or spirit of this invention. Upon reviewing the aforementioned embodiments, it would be evident to an artisan with ordinary skill in the art that said embodiments can be modified, reduced, or enhanced without departing from the scope and spirit of the claims described below.

We claim:

1. A system, comprising:
a memory that stores instructions; and
a processor that executes the instructions to perform operations, the operations comprising:
receiving, via an interface, individual search criteria associated with an individual;
generating a digital file including the individual search criteria associated with the individual;
formatting the digital file including the individual search criteria into a formatted digital file suitable for communication, storage, synthesis, analysis, or a combination thereof, by components of the system;
comparing the individual search criteria from the formatted digital file to information from a reference database;
identifying, based on the comparing, a potential relationship between the individual search criteria and a disease, condition, or a combination thereof, identified in the information from the reference database; and
presenting the potential relationship on a visualization interface on a device associated with the individual.

2. The system of claim 1, wherein the operations further comprise determining a degree of certainty of the potential relationship based on comparing the individual search criteria to aggregated information contained in a proprietary data warehouse, wherein the aggregated information comprises information corresponding to a plurality of individuals, a plurality of conditions, a plurality of scientific research data, a plurality of medical data, any type of data, or a combination thereof.

3. The system of claim 1, wherein the operations further comprise periodically querying the reference database and downloading relevant health data for future analyses to be conducted based on the individual search criteria, future individual search criteria, or a combination thereof.

4. The system of claim 1, wherein the operations further comprise updating a proprietary data warehouse by aggregating the individual search criteria, information associated with the potential relationship, information associated with an analysis conducted by the system on the potential relationship, metadata associated with the individual search criteria, or a combination thereof, with existing information in the proprietary data warehouse to generate updated data.

5. The system of claim 4, wherein the operations further comprise formatting the updated data for future re-use in additional system data analysis by-products.

6. The system of claim 1, wherein the individual search criteria comprises a keyword, a genomic signature of the individual, a search term, any type of criteria, a filter, or a combination thereof.

7. The system of claim 1, wherein the operations further comprise detecting a genetic anomaly associated with the individual based on comparing the individual search criteria from the formatted digital file to the information from the reference database.

8. The system of claim 1, wherein the operations further comprise initiating real-time monitoring of the individual based on the potential relationship identified.

9. The system of claim 1, wherein the operations further comprise determining a preventive action for mitigating or preventing the disease, the condition, or a combination thereof, associated with the potential relationship.

10. The system of claim 1, wherein the operations further comprise conducting a simulation for simulating the disease, the condition, or a combination thereof, associated with the potential relationship.

11. The system of claim 1, wherein the operations further comprise visually presenting the simulation to the individual via the visualization interface.

12. The system of claim 1, wherein the operations further comprise providing the potential relationship, an analysis of the potential relationship, the individual search criteria, metadata associated with the search criteria, or a combination thereof, to a device associated with a health professional for further analysis.

13. The system of claim 1, wherein the operations further comprise conducting a simulation of an outbreak, a population shift in health, an age progression, a disease progression, a condition progression, or a combination thereof.

14. A method, comprising:
receiving, via an interface, individual search criteria associated with an individual;
creating a digital file including the individual search criteria associated with the individual;
converting the digital file including the individual search criteria into a formatted digital file suitable for communication, storage, synthesis, analysis, or a combination thereof, by components of a system implementing the method;
comparing, by utilizing instructions from a memory that are executed by a processor, the individual search criteria from the formatted digital file to information from a reference database;
identifying, based on the comparing, a potential relationship between the individual search criteria and a disease, condition, or a combination thereof, identified in the information from the reference database; and
displaying the potential relationship on a visualization interface on a device associated with the individual.

15. The method of claim 14, further comprising training an artificial intelligence system of the system, a machine learning system of the system, or a combination thereof, based on the potential relationship, the individual search criteria, metadata associated with the potential relationship, metadata associated with the individual search criteria, or a combination thereof.

16. The method of claim 14, further comprising resetting the individual search criteria to generate a feedback look into the system so as to train an artificial intelligence system of the system, a machine learning system of the system, or a combination thereof.

17. The method of claim 14, further comprising comparing the individual search criteria to the information from the reference database by utilizing a mathematical algorithm.

18. The method of claim 14, further comprising enhancing a search algorithm, an analytics algorithm, or a combination thereof, utilized by the system based on the potential relationship, the individual search criteria, metadata associated with the potential relationship, metadata associated with the individual search criteria, or a combination thereof.

19. The method of claim 14, further comprising predicting an outcome associated with the individual based on the potential relationship identified.

20. A non-transitory computer-readable device comprising instructions, which when loaded and executed by a processor, cause the processor to perform operations comprising:
receiving, via an interface, individual search criteria associated with an individual;
generating a digital file including the individual search criteria associated with the individual;
converting the digital file including the individual search criteria into a formatted digital file suitable for communication, storage, synthesis, analysis, or a combination thereof, by components of a system implementing the method;
comparing the individual search criteria from the formatted digital file to information from a reference database;
identifying, based on the comparing, a potential relationship between the individual search criteria and a disease, condition, or a combination thereof, identified in the information from the reference database; and
presenting the potential relationship on a visualization interface on a device associated with the individual.
